(19) United States
(12) Patent Application Publication
(10) Pub. No.: US 2015/0251972 A1
(43) Pub. Date: Sep. 10, 2015

(54) PROCESS FOR REDUCING THE AMOUNT OF NORMAL PENTANE FROM A FEEDSTOCK

(71) Applicant: UOP LLC, Des Plaines, IL (US)

(72) Inventors: David James Shecterle, Arlington Heights, IL (US); Stephen Thomas King, Villa Park, IL (US); Xin X. Zhu, Des Plaines, IL (US)

(73) Assignee: UOP LLC, Des Plaines, IL (US)

(21) Appl. No.: 14/247,477

(22) Filed: Apr. 8, 2014

Related U.S. Application Data
(60) Provisional application No. 61/947,820, filed on Mar. 4, 2014.

(51) Int. Cl. C07C 5/27; C07C 7/04

(52) U.S. Cl. CPC C07C 5/2729; C07C 7/04

(57) ABSTRACT

A process for separation and treatment of a naphtha feedstock to increase overall octane in a gasoline blending pool by reducing or removing the normal pentane in the feedstock. The feedstock is passed into a divided wall column having an undivided top portion, an undivided bottom portion and a wall dividing a middle portion into two sections. The intermediate faction can include either all normal pentane, which can be utilized on other processes, or it can include a mixture of normal pentane and C₆ hydrocarbons, which can be isomerized in an isomerization zone to increase the octane and then passed to a gasoline blending pool.

### RELATED APPLICATIONS

This application claims priority to U.S. Provisional Application No. 61/947,820 filed on Mar. 4, 2014, the entirety of which is incorporated herein by reference.

### FIELD OF THE INVENTION

The present invention relates to a process for reducing the amount of normal pentane from a feedstock, and more specifically, to a process in which normal pentane is separated out from a feedstock alone or in combination with a C₆ hydrocarbon stream.

### BACKGROUND OF THE INVENTION

The naphtha boiling range hydrocarbons sold commercially as gasoline are normally a blend of several streams produced in a petroleum refinery. These include reformates and alkylates which are relatively sulfur free because of upstream refining. Another major source of the naphtha boiling range hydrocarbons is processing units which do not receive a highly de-sulfurized feed. These include hydrocracking units, coking units and fluidized catalytic cracking (FCC) process units. The refining industry is constantly seeking methods and process which increase the octane of its products. Many of these processes and methods utilize a diving wall fractionation column.

U.S. Pat. No. 2,471,134 illustrates a dividing wall fractionation column having a partition or dividing wall dividing the trayed column into two parallel vapor-liquid contacting chambers. A similar but more detailed disclosure of a dividing wall fractionation column is provided by U.S. Pat. No. 4,230,533. Dividing wall columns are closely related to a different type of column referred to as a partitioned distillation column such as illustrated in U.S. Pat. No. 5,755,933. A partitioned distillation column differs from a dividing wall column in that the vertical dividing wall is positioned such that it contacts one end of the column. Thus, only one terminal portion of the column is divided into the two parallel contacting sections. In this manner two overhead products or two bottom products may be removed from a single column.

U.S. Pat. No. 6,927,314 discloses the use of a dividing wall column in a process to increase the octane of a naphtha feedstock in which the dividing wall column is used to separate a feedstock to three streams, a overhead stream, an intermediate fraction, and a bottoms stream. The overhead stream comprises C₅ hydrocarbons. The intermediate fraction comprises C₆ hydrocarbons. The bottoms stream comprises C₇+ hydrocarbons. The intermediate fraction is passed through an isomerization zone to increase the octane of same before being passed to a gasoline blending pool. The overhead stream is passed directly to the gasoline blending pool without further fractionization or isomerization of the overhead stream. While it is recognized that the overhead stream includes normal pentane (“n-pentane”), it is disclosed that the amount is minimal. It is also disclosed that isomerizing the overhead stream (C₅ hydrocarbons) can actually lower the octane number.

However, n-pentane has an undesirably low octane number. Thus, inclusion of n-pentane in a gasoline blending pool will lower the overall octane of the resulting gasoline. Additionally, the inclusion of n-pentane will increase the vapor pressure of the gasoline blend, which is undesirable.

Accordingly, known processes typically use distillation columns for both pre-fractionation of the feed and post fractionation of the reactor products. Depending on a refiner's gasoline blending requirements, isomerization unit pre-fractionation schemes can include columns which separate an isopentane rich stream or n-pentane rich stream. However, these columns take up a lot of plot space and have both high capital expenditures and high operating expenditures.

Therefore, it would be desirable to have an efficient process to either remove n-pentane from the feedstock, minimize the amount of same in an efficient manner, or both.

### SUMMARY OF THE INVENTION

A first embodiment of the invention may be characterized as a process for the separation and treatment of a naphtha feedstock comprising compounds containing from five to six carbon atoms in which: the feedstock is passed into a separation zone comprising a column divided into at least a first fractionization zone and a second fractionization zone by a dividing wall, the first fractionization zone being parallel to the second fractionization zone, each fractionization zone having an upper end and a lower end, the upper ends of the fractionization zones being in open communication at an undivided upper section of the column, and the lower ends of the fractionization zones being in open communication at an undivided lower section of the column, and the feedstock entering into the column in the first fractionization zone; the feedstock is separated into: an overhead stream comprising isopentane; an intermediate fraction comprising n-pentane; and, a bottoms stream comprising compounds containing six or more carbon atoms; the overhead stream is recovered from the upper section of the column; the overhead stream is passed to a gasoline blending pool; the intermediate fraction is recovered from the second fractionization zone; the bottoms stream is recovered from the lower section of the column; the bottoms stream is passed to an isomerization zone to increase the octane number of the bottoms stream and form an isomerate; and, the isomerate is passed to the gasoline blending pool.

A second embodiment of the invention may be characterized as a process for the separation and treatment of a naphtha feedstock comprising compounds containing from five to seven or more carbon atoms in which: the feedstock is passed into a separation zone comprising a column having an undivided upper section, an undivided lower section, and a intermediate section disposed between the upper section and lower section, the intermediate section divided with a wall into a first intermediate zone and a second intermediate zone; the feedstock is separated into: an overhead stream comprising isopentane; an intermediate fraction comprising n-pentane and compounds containing six carbon atoms; and, a bottoms stream comprising compounds containing seven or more carbon atoms; the overhead stream is recovered from the upper section of the column; the overhead stream is passed to a gasoline blending pool; the intermediate fraction is recovered from the second intermediate zone; the intermediate fraction is treated in an isomerization zone to increase the octane number of the intermediate fraction and form an isomerate; the isomerate is passed to a gasoline blending pool; and, the bottoms stream is recovered from the lower section of the column.

Another embodiment of the invention may be characterized as a process for the separation and treatment of a naphtha feedstock comprising compounds containing from five to seven or more carbon atoms in which: the feedstock is passed into a separation zone comprising a column having an undivided upper section, an undivided lower section, and a intermediate section disposed between the upper section and lower section, the intermediate section divided with a wall into a first intermediate zone and a second intermediate zone; the feedstock is separated into: an overhead stream comprising isopentane; an intermediate fraction comprising either n-pentane or a mixture of n-pentane and compounds containing six carbon atoms; and, a bottoms stream comprising either compounds containing six carbon atoms or more if the intermediate fraction is rich in n-pentane or compounds containing seven or more carbon atoms if the intermediate fraction includes the mixture of n-pentane and compounds containing six carbon atoms; the overhead stream is passed to a gasoline blending pool; the fraction which includes paraffin compounds containing six carbon atoms is passed in an isomerization zone to increase the octane number of the fraction and form an isomerate; and, the isomerate is passed to the gasoline blending pool.

In one or more embodiments of the present invention, the n-pentane has been removed from the feedstock. Thus, the other fractionated portions may be passed to processing units without the normal pentane. Thus, the overall octane of the gasoline blending pool may be increased.

In some embodiments of the present invention, the n-pentane is removed from the feedstock in a stream combined with the C₆ hydrocarbons. This stream can be sent to an isomerization unit, which will provide a stream with a higher octane. However, since the isopentane has already been separated, the isomerization of the n-pentane will not result in a lowering of the octane number.

### DETAILED DESCRIPTION OF THE DRAWINGS

The drawings are simplified process flow diagrams showing the fractionation of a full boiling range naphtha into light, heavy and intermediate boiling range fractions using a divided wall column to reduce the amount of n-pentane from the feed stream.

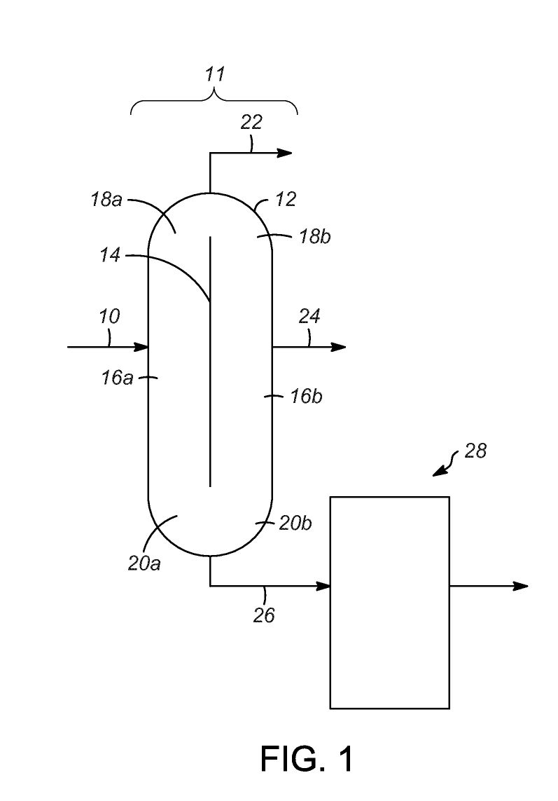

FIG. 1 shows a process flow diagram in which a divided wall column is used to isolate and recover an intermediate stream of n-pentane.

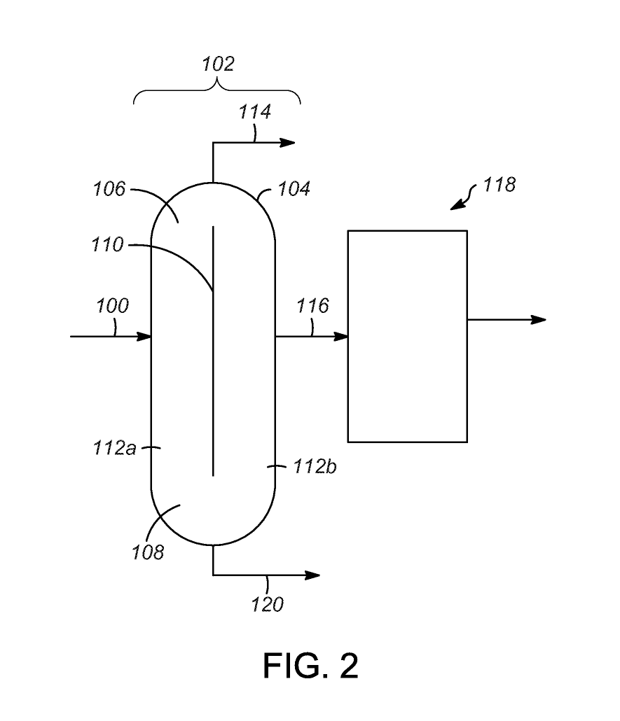

FIG. 2 shows a process flow diagram of in which a divided wall column is used to isolate and recover an intermediate stream of a mixture of n-pentane and C₆ hydrocarbons.

### DETAILED DESCRIPTION OF THE INVENTION

The feedstock or streams utilized in the present invention are naphtha boiling range petroleum fractions such as FCC gasoline, coker naphtha, straight run gasoline and naphtha fractions from conversion processes such as hydrocracking or thermal cracking. These gasoline blending component streams will normally have a boiling range, as determined by the appropriate ASTM test method, falling between about 38° C. to 260° C. (100° F. and 500° F.), which encompasses the range of boiling points for modern gasoline. The individual feeds may include a light naphtha having a boiling point range of from that of C₅ to about 96° C. (205° F.), full range naphtha having a boiling point range from about that of C₅ hydrocarbons to about 204° C. (400° F.) and heavy naphtha boiling fraction distilling in the range of from about 96° C. to about 204° C. (about 205° F. to about 400° F.).

The present invention provides a process to increase the octane of a stream destined for a gasoline blending pool by minimizing or reducing the n-pentane from a feedstock. The separation results in three streams, a lighter stream, an intermediate stream and a heavy stream. The lighter stream is rich in isopentane and may be optionally treated to remove sulfur and then directed to a gasoline pool.

In some embodiments of the present invention, the intermediate stream contains C₆ hydrocarbons and n-pentane. In these embodiments, the intermediate stream may also be optionally treated to remove sulfur, and then it may be passed to an isomerization zone to convert low octane components into higher octane, more valuable, components. The higher octane isomerate may then be passed to the gasoline pool.

In some embodiments of the present invention the intermediate stream is rich in n-pentane. In these embodiments, the intermediate stream may be stored, or sent to some other process. Furthermore, in such embodiments, the heavy stream will most likely contain C₆+ hydrocarbons. Thus, the heavy stream may also be optionally treated to remove sulfur, and then is passed to an isomerization zone to convert low octane components into higher octane, more valuable, components. The higher octane isomerate may then be passed to the gasoline pool, or the heavy stream may be further fractionalized to separate the C₆ hydrocarbons from the heavier components.

Not only does the invention provide a process for increasing the octane of a stream, but the invention also provides a particular fractionation design that is surprisingly efficient from both a fixed cost perspective as well as a utilities perspective.

The invention will be explained in detail where the feed is a light naphtha containing approximately 0.8 mass % C₄ hydrocarbons, 53 mass % C₅ hydrocarbons, 46 mass % C₆ hydrocarbons, and 0.2 mass % C₇+ hydrocarbons. It is to be understood however, that other naphthas having different ranges of components may also be separated according to the present invention, and the octane of the overall result be enhanced.

The C₅ hydrocarbons of FCC gasoline include multi-methyl branched pentane, isopentane, and n-pentane. As discussed above, isomerizing the C₅ hydrocarbons typically found would not increase the octane number and in fact may decrease the octane number by isomerizing some of the high octane components to lower octane components. The C₅ hydrocarbons of the FCC gasoline typically have an octane number of about 93, and isomerizing the C₅ hydrocarbons fraction may actually decrease the octane number to about 91. However, in prior art processes, the entire C₅ hydrocarbons fraction would be passed to a gasoline blending pool.

In comparison, the C₆ hydrocarbons are largely normal and mono-methyl branched hydrocarbons which have lower octane numbers, such as between approximately 50 to approximately 70. After isomerization to form multi-methyl branched C₆ hydrocarbons, the octane number may be increased to between approximately 60 to approximately 91. This is a sizeable increase in octane number for this fraction of the FCC gasoline.

As to the C₇+ hydrocarbons of the FCC gasoline, most isomerization processes successful for the isomerization of C₆ hydrocarbons have a tendency to crack larger carbon number hydrocarbons. The cracked product has a lesser value; therefore, it is not desirable to isomerize the C₇+ hydrocarbons using the same isomerization system as for the C₆ hydrocarbons. Also, some gasoline yield loss may occur due to the cracking forming lighter products.

The inclusion of n-pentane in the gasoline blending pool has been found to increase the vapor pressure of the resultant gasoline while at the same time lowering the overall octane of same. Therefore, it is desirable to minimize the n-pentane that is supplied to the gasoline blending pool. The difficulty lies in the fact that the n-pentane component is found in the middle of the complete boiling point range of the FCC gasoline. Accordingly, three streams must be separated, a lighter stream, an intermediate stream, and a heavy stream.

A first embodiment of the present invention is shown in FIG. 1 in which a feedstock containing a mixture of C₅ through C₇+ hydrocarbons in line 10 enters a separation zone 11 including at least a dividing wall main fractionation column 12. The depiction of column 12 is simplified as all the auxiliary operational components, such as controls, trays, condenser and reboiler, may be of conventional design. In other embodiments, different stocks can be fed into column 12 at different locations if appropriate. The dividing wall column 12 is distinguished from some traditional fractional columns by the presence of a vertical dividing wall 14 in a vertical mid portion of the column 12, also referred to as the dividing wall portion of the column 12.

This dividing wall 14 extends between opposing sides of the inner surface of the column 12 and joins it in a substantially fluid tight seal. Thus, fluids cannot pass horizontally from one side of the column 12 to the other and must instead travel either over or under the wall 14. The dividing wall 14 divides the central portion of the column 12 into two parallel fractionation zones or chambers 16a, 16b, which may be of different cross-section. Each chamber 16a, 16b and the rest of the column 12 will contain conventional vapor liquid contacting equipment such as trays or packing. The type of tray and design details such as tray type, tray spacing and layout may vary within the column 12 and between the two parallel chambers 16a, 16b of the dividing wall portion of the column 12.

Additionally, as shown, each chamber 16a, 16b has an upper end 18a, 18b, and a lower end 20a, 20b. Since the dividing wall 14 is present only in the middle of the column 12, the upper ends 18a, 18b of the two chambers 16a, 16b are in open communication. Additionally, the lower ends 20a, 20b of the two chambers 16a, 16b are likewise in open communication.

In this embodiment of the present invention, the dividing wall column 12 separates all of the entering naphtha boiling range hydrocarbons into an overhead stream being rich in isopentane, an intermediate (or side draw) stream being rich in n-pentane, and a bottoms stream containing the heavier C₆+ hydrocarbons. As will be appreciated by those of ordinary skill in the art, when separating hydrocarbons, there typically can be some crossover between the various fractions/streams during the separation processes and thus, the present invention is intended to accommodate the crossover amounts of compounds.

The overhead (or light) fraction, rich in isopentane, is removed from the column 12 via a line 22. As isopentane already has a satisfactorily high octane number, the overhead fraction in line 22 may be passed to a gasoline blending pool.

The intermediate fraction, rich in n-pentane, is removed from the column 12 via a line 24. Since the overhead fraction already provides the gasoline blending pool with a sufficient amount of C₅ hydrocarbons, it is contemplated that the intermediate fraction in this embodiment of the invention is utilized in another process. For example, n-pentane is desirable as a feed for cracking process, such as in a steam cracker, for the production of olefins. Thus, the intermediate fraction may be stored prior to use.

Furthermore, since the n-pentane, which has a lower octane number, is not sent to the gasoline blending pool, the octane of the resulting gasoline blending pool will be increased compared to the resulting blend if n-pentane had been sent to the gasoline blending pool.

The bottoms stream (or heavy fraction) is removed from the column 12 via a line 26 and comprises C₆ or C₆+ hydrocarbons. The bottoms stream can be sent to an isomerization zone 28. If the bottoms stream comprises C₇+ hydrocarbons, the bottoms stream may first pass through a separation zone to separate the bottoms stream into a C₆ stream and a C₇+ stream.

In the isomerization zone, the bottoms stream fraction is preferably contacted with an isomerization catalyst under conditions which effect the isomerization of the lower octane number components into higher octane number components. The isomerate may be passed to the gasoline blending pool. The details of the isomerization zone are known in the art and are not necessary for one of ordinary skill in the art to practice the embodiments of the present invention.

It was believed that at least about 90% of the total isopentane from the feedstock could be recovered in process. Additionally, it is also believed that at least about 80% of the total n-pentane in the feedstock, and most preferably at least about 90% of the total n-pentane in the feedstock, could be recovered in such a process.

A theoretical modeling was conducted for the embodiment of the invention shown in FIG. 1 in which the intermediate stream is an n-pentane rich stream. For this theoretical modeling the feed rate was 385 std m³/h (58,050 BPSD). The reboiler duty was 34.7 MMkcal/h (138 MM BTU/hr). The intermediate stream had a flow rate of 125 std m³/h (18,900 BPSD). The temperature and pressure of the dividing wall column were 58° C. (136° F.) and 276 KPaa (40 Psia), respectively, and were measured at the overhead receiver. The results of this theoretical model are shown in the below TABLE 1.

TABLE 1
n-PENTANE RICH INTERMEDIATE STREAM

| | | | | |
|---|---|---|---|---|
| Flow Rates (kg/hr) | 250,907 | 51,297 | 79,296 | 120,907 |
| Flow Rates (lb/hr) | 553,149 | 113,089 | 174,817 | 265,243 |
| Mass Fractions | Feed | Overhead | Intermediate | Bottoms |
| C₄ | 0.0085 | 0.0418 | 0.0000 | 0.0000 |
| NEOPENTANE | 0.0029 | 0.0141 | 0.0000 | 0.0000 |
| ISOPENTANE | 0.2849 | 0.8919 | 0.3156 | 0.0059 |
| n-PENTANE | 0.2191 | 0.0458 | 0.6172 | 0.0305 |
| CYCLOPENTANE | 0.0196 | 0.0000 | 0.0297 | 0.0213 |
| 1-PENTENE | 0.0025 | 0.0064 | 0.0037 | 0.0001 |
| C₆ | 0.4602 | 0.0000 | 0.0338 | 0.9375 |
| C₇ | 0.0023 | 0.0000 | 0.0000 | 0.0047 |

As shown, approximately 90% of the n-pentane of the feedstock was separated into the intermediate stream. Furthermore, approximately 64% of the isopentane of the feedstock was separated into the overhead stream. With a lower purity, approximately 90% of the isopentane may be separated. Thus, a significant amount of the n-pentane of the feedstock can be separated from the remaining components.

Another embodiment of the present invention is shown in FIG. 2, in which the feedstock may again contain a mixture of C₅ through C₇+ hydrocarbons. Through a line 100, the feedstock enters a separation zone 102 which may include a dividing wall main fractionation column 104. The depiction of column 104 is again simplified as all the auxiliary operational components, such as controls, trays, condenser and reboiler, may be of conventional design. However, as will be appreciated based upon the following discussion, the trays and other internal components will vary based upon the desired separations.

The dividing wall column 104 includes an undivided upper section 106, an undivided lower section 108, and a wall 110 separating the middle section of the column 104 into a first intermediate zone 112a and a second intermediate zone 112b. The column 104 separates all of the entering naphtha boiling range hydrocarbons into an overhead stream being rich in isopentane, an intermediate side draw stream containing a mixture of n-pentane and C₆ hydrocarbons, and a bottom stream containing the heavier C₇+ hydrocarbons. Again, as will be appreciated by those of ordinary skill in the art, when separating hydrocarbons, there typically can be some crossover between the various fractions/streams during the separation processes and thus, the present invention is intended to accommodate the crossover amounts of compounds.

As shown in FIG. 2, the overhead fraction, rich in isopentane, is removed from the column 104 via a line 114. Again, since isopentane already has a high octane number, the overhead fraction in the line 114 may be passed to a gasoline blending pool.

The intermediate fraction, a mixture of n-pentane and C₆ hydrocarbons, is removed from the column 104 via a line 116. In this embodiment of the present invention, the intermediate fraction may be sent to an isomerization zone 118.

In the isomerization zone 118, the intermediate fraction is preferably contacted with an isomerization catalyst under conditions which effect the isomerization of the lower octane number components into higher octane number components. The isomerate may be passed to the gasoline blending pool. Again, the details of the isomerization zone 118 are not discussed in detail herein.

The bottoms stream fraction is removed from column 104 via line 120 and is rich in C₇₊ hydrocarbons. The bottoms stream fraction may be passed to gasoline blending pool or may be passed to a reforming zone, to produce a reformate, and the reformate may be passed to the gasoline blending pool.

If n-pentane has been combined with the C₆ hydrocarbons in the intermediate fraction, it has been found that designing the column 104 such that benzene, methylcyclopentane, and cyclohexane are removed in the intermediate fraction provides for lower benzene content in the gasoline pool. However, it is also contemplated the benzene, methylcyclopentane, and cyclohexane are contained with the bottoms stream, resulting in maximum benzene production and the processes of the present invention would still provide acceptable results for the purposes of the present invention.

Therefore, in embodiments in which the amount of benzene and benzene precursors such as cyclohexane in the intermediate fraction were minimized, it believed that at least about 70%, most preferably at least 90%, of the isopentane in the feedstock can be separated from the other components of the feedstock. In such embodiments, it is believed that between about 85% to about 90% of the total n-pentane in the feedstock, and approximately about 99% of the total C₆ paraffin hydrocarbons could be separated from the other components in the feedstock.

Additionally, in embodiments in which the amount of benzene in the intermediate fraction was not minimized, it is believed that at least about 80%, and preferably at least 90%, and most preferably approximately 95% of the isopentane in the feedstock can be separated from the other components of the feedstock. In such embodiments, it is also believed that at least about 60% to 90% of the total n-pentane in the feedstock, and approximately 90% to 99% of the total C₆ hydrocarbons in the feedstock can be separated from the other components in the feedstock.

In any of these embodiments in which the intermediate fraction includes n-pentane and C₆ hydrocarbons, since the n-pentane was separated from the feedstock along with the C₆ hydrocarbons, when the n-pentane is isomerized to increase octane, the overall octane of the n-pentane will increase. Thus, the overall octane in the gasoline blending pool has also increased.

A second theoretical modeling was conducted for the embodiment of the present invention shown in FIG. 2 in which the intermediate stream comprises a mixture of n-pentane and C₆ hydrocarbons. For this theoretical modeling, the feed rate was 862 std m³/h (130,079 BPSD). The reboiler duty was 119 MMkcal/h (472 MM BTU/hr). The intermediate stream had a flow rate of 283 std m³/h (42,747 BPSD). The temperature and pressure of the dividing wall column were 60° C. (140° F.) and 276 KPaa (40 Psia), respectively, and were measured at the overhead receiver. The results of this theoretical model are shown in the below TABLE 2.

TABLE 2
n-PENTANE AND C₆ INTERMEDIATE STREAM

| | | | | |
|---|---|---|---|---|
| Flow Rate (kg/hr) | 609,337 | 81,665 | 194,545 | 333,127 |
| Flow Rate (lb/hr) | 1,343,345 | 180,037 | 428,895 | 734,413 |
| Mass Fractions | Feed | Overhead | Intermediate | Bottoms |
| C₄ | 0.0035 | 0.0259 | 0.0000 | 0.0000 |
| NEOPENTANE | 0.0012 | 0.0089 | 0.0000 | 0.0000 |
| ISOPENTANE | 0.1077 | 0.7506 | 0.0223 | 0.0000 |
| n-PENTANE | 0.0852 | 0.2147 | 0.1766 | 0.0000 |
| CYCLOPENTANE | 0.0054 | 0.0000 | 0.0170 | 0.0000 |
| C₆ | 0.2659 | 0.0000 | 0.7672 | 0.0383 |
| C₇ | 0.3090 | 0.0000 | 0.0169 | 0.5554 |
| C₈ | 0.2221 | 0.0000 | 0.0000 | 0.4063 |

As shown, approximately 60% of the n-pentane of the feedstock was separated into the intermediate stream. Furthermore, at least 90% of the isopentane of the feedstock was separated into the overhead stream. Thus, a significant amount of the n-pentane of the feedstock can be combined with the C₆ stream.

In an effort to make the different theoretical modelings of TABLES 1 and 2 comparable, both of the flowschemes included approximately 85 separatory stages. However, it should be understood that is merely exemplary, and that separation zones with different numbers of stages can be used.

In TABLE 3, shown below, a theoretical modeling was done in which the intermediate stream contained n-pentane, C6 paraffins, and in which the amount of benzene and benzene precursors was minimized.

TABLE 3
MINIMIZE BENZENE PRECURSORS IN INTERMEDIATE

| | | | | |
|---|---|---|---|---|
| Flow Rate (kg/hr) | 609,337 | 100,380 | 135,667 | 373,290 |
| Flow Rate (lb/hr) | 1,343,345 | 221,298 | 299,091 | 822,955 |
| Mass Fractions | Feed | Overhead | Intermediate | Bottoms |
| C₄ | 0.0035 | 0.0210 | 0.0000 | 0.0000 |
| NEOPENTANE | 0.0012 | 0.0072 | 0.0000 | 0.0000 |
| ISOPENTANE | 0.1077 | 0.5718 | 0.0607 | 0.0000 |
| n-PENTANE | 0.0852 | 0.3999 | 0.0866 | 0.0000 |
| 1-PENTENE | 0.0000 | 0.0000 | 0.0000 | 0.0000 |
| CYCLOPENTANE | 0.0054 | 0.0000 | 0.0243 | 0.0000 |
| 2,2-DIMETHYLBUTANE | 0.0056 | 0.0000 | 0.0253 | 0.0000 |
| 2,3-DIMETHYLBUTANE | 0.0094 | 0.0000 | 0.0423 | 0.0000 |
| 2-METHYLPENTANE | 0.0519 | 0.0000 | 0.2331 | 0.0000 |
| 3-METHYLPENTANE | 0.0321 | 0.0000 | 0.1423 | 0.0006 |
| n-HEXANE | 0.0736 | 0.0000 | 0.3133 | 0.0062 |
| 1-HEXENE | 0.0000 | 0.0000 | 0.0000 | 0.0000 |
| METHYLCYCLOPENTANE | 0.0278 | 0.0000 | 0.0155 | 0.0398 |
| CYCLOHEXANE | 0.0249 | 0.0000 | 0.0046 | 0.0390 |
| BENZENE | 0.0405 | 0.0000 | 0.0278 | 0.0561 |
| C₇ | 0.5312 | 0.0000 | 0.0242 | 0.8582 |

In TABLE 4, shown below, a theoretical modeling was done in which the intermediate stream contained n-pentane, C₆ paraffins, and in which the amount of benzene and benzene precursors was maximized while also minimizing the C₇ fraction to be approximately 3%.

TABLE 4
MAXIMIZE BENZENE PRECURSORS IN INTERMEDIATE

| | | | | |
|---|---|---|---|---|
| Flow Rate (kg/hr) | 609,337 | 64,388 | 226,037 | 318,914 |
| Flow Rate (lb/hr) | 1,343,345 | 141,950 | 498,320 | 703,077 |
| Mass Fractions | Feed | Overhead | Intermediate | Bottoms |
| C₄ | 0.0035 | 0.0328 | 0.0000 | 0.0000 |
| NEOPENTANE | 0.0012 | 0.0108 | 0.0001 | 0.0000 |
| ISOPENTANE | 0.1077 | 0.7396 | 0.0797 | 0.0000 |
| n-PENTANE | 0.0852 | 0.2167 | 0.1679 | 0.0000 |
| 1-PENTENE | 0.0000 | 0.0000 | 0.0000 | 0.0000 |
| CYCLOPENTANE | 0.0054 | 0.0000 | 0.0146 | 0.0000 |
| 2,2-DIMETHYLBUTANE | 0.0056 | 0.0000 | 0.0152 | 0.0000 |
| 2,3-DIMETHYLBUTANE | 0.0094 | 0.0000 | 0.0254 | 0.0000 |
| 2-METHYLPENTANE | 0.0519 | 0.0000 | 0.1399 | 0.0000 |
| 3-METHYLPENTANE | 0.0321 | 0.0000 | 0.0865 | 0.0000 |
| n-HEXANE | 0.0736 | 0.0000 | 0.1983 | 0.0001 |
| 1-HEXENE | 0.0000 | 0.0000 | 0.0000 | 0.0000 |
| METHYLCYCLOPENTANE | 0.0278 | 0.0000 | 0.0748 | 0.0002 |
| CYCLOHEXANE | 0.0249 | 0.0000 | 0.0620 | 0.0037 |
| BENZENE | 0.0405 | 0.0000 | 0.1091 | 0.0001 |
| C₇ | 0.5312 | 0.0000 | 0.0266 | 0.9960 |

Based upon any of these embodiments of the present invention, a reduction of the n-pentane in the feedstock provides for a higher octane gasoline blend. In addition to the realized increase in octane number, there are also savings in plot space and capital expenditures when using a single tower. Furthermore, product purities may also be increased when going to a single dividing wall tower with side draw as more stages may be utilized to allow for better separation of the fractionization streams.

Therefore, as will be appreciated, a process according to one or more of these embodiments provides an effective and efficient method to separate n-pentane from a feedstock.

As is apparent from the foregoing specification, the invention is susceptible of being embodied with various alterations and modifications which may differ particularly from those that have been described in the preceding specification and description. It should be understood that we wish to embody within the scope of the patent warranted hereon all such modifications as reasonably and properly come within the scope of our contribution to the art.

What is claimed is:

1. A process for the separation and treatment of a naphtha feedstock comprising compounds containing from five to six carbon atoms, said process comprising:
passing the feedstock into a separation zone comprising a column divided into at least a first fractionization zone and a second fractionization zone by a dividing wall, the first fractionization zone being parallel to the second fractionization zone, each fractionization zone having an upper end and a lower end, the upper ends of the fractionization zones being in open communication at an undivided upper section of the column, and the lower ends of the fractionization zones being in open communication at an undivided lower section of the column, and the feedstock entering into the column in the first fractionization zone;
separating the feedstock into: an overhead stream comprising isopentane; an intermediate fraction comprising n-pentane; and, a bottoms stream comprising compounds containing six or more carbon atoms;
recovering the overhead stream from the upper section of the column;
passing the overhead stream to a gasoline blending pool;
recovering the intermediate fraction from the second fractionization zone;
recovering the bottoms stream from the lower section of the column;
passing the bottoms stream to an isomerization zone to increase the octane number of the bottoms stream and form an isomerate; and,
passing the isomerate to the gasoline blending pool.

2. The process of claim 1 further comprising:
passing the intermediate fraction to a cracking zone.

3. The process of claim 2 wherein the cracking zone comprises a steam cracker.

4. The process of claim 1 wherein the overhead stream comprises at least 60% of a total isopentane in the feedstock.

5. The process of claim 1 wherein the overhead stream comprises at least 90% of the total isopentane in the feedstock.

6. The process of claim 1 wherein the intermediate fraction comprises at least 80% of a total n-pentane in the feedstock.

7. The process of claim 1 wherein the overhead stream comprises at least 60% isopentane of a total isopentane in the feedstock and the intermediate fraction comprises at least 90% of a total n-pentane in the feedstock.

8. The process of claim 1 further comprising:
storing the intermediate fraction.

9. The process of claim 1 wherein the feedstock is selected from the group consisting of: FCC gasoline, coker naphtha, straight run naphtha, naphtha fraction from a hydrocracking process, naphtha fraction from a thermal cracking process and mixtures thereof.

10. A process for the separation and treatment of a naphtha feedstock comprising compounds containing from five to seven or more carbon atoms, said process comprising:
passing the feedstock into a separation zone comprising a column having an undivided upper section, an undivided lower section, and an intermediate section disposed between the upper section and lower section, the intermediate section divided with a wall into a first intermediate zone and a second intermediate zone;
separating the feedstock into: an overhead stream comprising isopentane; an intermediate fraction comprising n-pentane and compounds containing six carbon atoms; and, a bottoms stream comprising compounds containing seven or more carbon atoms;
recovering the overhead stream from the upper section of the column;
passing the overhead stream to a gasoline blending pool;
recovering the intermediate fraction from the second intermediate zone;
treating the intermediate fraction in an isomerization zone to increase the octane number of the intermediate fraction and form an isomerate;
passing the isomerate to a gasoline blending pool; and,
recovering the bottoms stream from the lower section of the column.

11. The process of claim 10 further comprising:
passing the bottoms stream to a reforming zone to create a reformate; and,
passing the reformate to the gasoline blending pool.

12. The process of claim 10 wherein the intermediate fraction is benzene rich and the overhead stream comprises at least about 70% of a total isopentane in the feedstock.

13. The process of claim 10 wherein the bottoms stream is benzene rich and the overhead stream comprises at least about 80% of a total isopentane in the feedstock.

14. The process of claim 10 wherein the intermediate fraction is benzene rich and the intermediate fraction comprises at least approximately 70% of a total n-pentane in the feedstock.

15. The process of claim 10 wherein the intermediate fraction includes approximately 99% of an amount of paraffin compounds containing six carbon atoms in the feedstock.

16. The process of claim 10 wherein the bottoms stream is benzene rich and the intermediate fraction comprises at least approximately 20% of a total n-pentane in the feedstock.

17. The process of claim 16 wherein the intermediate faction comprises approximately 97% of an amount of paraffin compounds containing six carbon atoms in the feedstock.

18. A process for the separation and treatment of a naphtha feedstock comprising compounds containing from five to seven or more carbon atoms, said process comprising:
passing the feedstock into a separation zone comprising a column having an undivided upper section, an undivided lower section, and an intermediate section disposed between the upper section and lower section, the intermediate section divided with a wall into a first intermediate zone and a second intermediate zone;
separating the feedstock into: an overhead stream comprising isopentane; an intermediate fraction comprising either n-pentane or a mixture of n-pentane and compounds containing six carbon atoms; and, a bottoms stream comprising either compounds containing six carbon atoms or more if the intermediate fraction is rich in n-pentane or compounds containing seven or more carbon atoms if the intermediate fraction includes the mixture of n-pentane and compounds containing six carbon atoms;
passing the overhead stream to a gasoline blending pool;
treating the fraction which includes compounds containing six carbon atoms in an isomerization zone to increase the octane number of the fraction and form an isomerate; and,
passing the isomerate to the gasoline blending pool.

19. The method of claim 18 wherein the overhead stream includes at least 70% of a total isopentane in the feedstock.

20. The method of claim 19 wherein the intermediate fraction includes at least 20% of a total n-pentane in the feedstock.
